(19) United States
(12) Patent Application Publication
(10) Pub. No.: US 2025/0228656 A1
(43) Pub. Date: Jul. 17, 2025

(54) AUTOMATIC HAIR TRANSPLANT DEVICE

(71) Applicant: KANEKA CORPORATION, Osaka-shi, Osaka (JP)

(72) Inventors: Kai TACHIMORI, Toyooka-shi (JP); Kenta INOUE, Toyooka-shi (JP); Terumitsu ODA, Takasago-shi (JP)

(21) Appl. No.: 18/848,794

(22) PCT Filed: Mar. 14, 2023
(86) PCT No.: PCT/JP2023/009808

§ 371 Date: Sep. 19, 2024

(30) Foreign Application Priority Data
Mar. 23, 2022 (JP) 2022-047360

(51) Int. Cl. A61F 2/10; A61F 2/00

(52) U.S. Cl. CPC A61F 2/10; A61F 2/0095

(57) ABSTRACT

Provided is an automatic hair transplant device capable of stably taking out a hair material from a storage container and transplanting the hair material. The automatic hair transplant device includes a storage container having an outlet at a bottom thereof, the storage container placing a hair material across the outlet on the bottom. Furthermore, the automatic hair transplant device includes an energizing part configured to apply a force to the hair material, the force having a component in a direction away from the outlet.

### TECHNICAL FIELD

The present invention relates to an automatic hair transplant device for transplanting a hair material into a hair transplant net.

### BACKGROUND ART

Patent Document 1 proposes an automatic hair transplant device that ties a hair material to a hair transplant net to transplant the hair material. This automatic hair transplant device includes a storage container in which a hair material bundle including a plurality of hair materials is housed and which has an outlet at a bottom. A hook needle is inserted into the outlet and then pulled out of the outlet, whereby the hair material caught by the hook needle is taken out and the hair material is tied to a hair transplant net.

### PRIOR ART DOCUMENT

### Patent Document

Patent Document 1: JP6533350B1

### DISCLOSURE OF INVENTION

### Technical Problem

In the automatic hair transplant device proposed in Patent Document 1, when the hook needle is inserted into the outlet, the hair material is pushed up by the hook needle at the outlet. At this time, not only the hair material directly brought into contact with the hook needle to be pushed up, but also the surrounding hair material is pushed up together by friction. In addition, when the hair material caught by the hook needle in the storage container is pulled out from the outlet, the surrounding hair material is also pulled downward together by the frictional force. Further, the hair material receives a force not only in the up-down direction but also in a direction on a horizontal plane.

As described above, the density of the hair materials in the vicinity of the outlet of the storage container is high enough to be caught by the hook needle at the beginning of installation, but as the hair material is taken out from the storage container, the hair material gradually warps or slackens, and thereby the density decreases. Eventually, it becomes difficult for the hook needle inserted into the outlet to catch and take out the hair material, thereby the hair material becoming unable to be transplanted onto the hair transplant net.

The present invention has been made to solve the above problems, and provides an automatic hair transplant device capable of stably taking out a hair material from a storage container and transplanting the hair material.

### Solution to Problem

In order to achieve the above object, an automatic hair transplant device including a storage container having an outlet at a bottom thereof, the storage container placing hair material across the outlet on the bottom; and an energizing part configured to apply a force to the hair material placed across the outlet on the bottom of the storage container, the force having a component in a direction away from the outlet.

### Effect of Invention

According to the automatic hair transplant device, bending and slackening of the hair material are alleviated by pulling the hair material in a direction away from the outlet, and reduction in density of the hair materials in the vicinity of the outlet in the storage container is improved. Therefore, according to the automatic hair transplant device, the hair material can be stably taken out from the storage container and transplanted.

### BRIEF DESCRIPTION OF DRAWINGS

FIG. 1 is a side view of an automatic hair transplant device 120.

FIG. 2 is a front view of the automatic hair transplant device 120.

FIG. 3 is a side view of the automatic hair transplant device 120.

FIG. 4 is a side view of the automatic hair transplant device 120.

FIG. 5 is a perspective view showing a configuration of a first example of a first embodiment.

FIG. 6 is a perspective view showing the configuration of the first example of the first embodiment.

FIG. 7 is a perspective view showing the configuration of the first example of the first embodiment.

FIG. 8 is a perspective view showing the configuration of the first example of the first embodiment.

FIG. 9 is a perspective view showing a configuration of a second example of the first embodiment.

FIG. 10 is a perspective view showing a configuration of the second example of the first embodiment.

FIG. 11 is a perspective view showing a configuration of a modification of the first example of the first embodiment.

FIG. 12 is a perspective view showing the configuration of the modification of the first example of the first embodiment.

FIG. 13 is a perspective view showing the configuration of the modification of the first example of the first embodiment.

FIG. 14 is a perspective view showing the configuration of the modification of the first example of the first embodiment.

FIG. 15 is a perspective view showing a configuration of a first example of a second embodiment.

FIG. 16 is a cross-sectional view taken along line A-A in FIG. 15.

FIG. 17 is a cross-sectional view showing a configuration of a second example of the second embodiment.

FIG. 18 is a cross-sectional view showing a configuration in which the first example and the second example of the second embodiment are combined.

FIG. 19 is a cross-sectional view showing a configuration of a modification of the second example of the second embodiment.

FIG. 20 is a cross-sectional view showing a configuration of a modification of the second example of the second embodiment.

FIG. 21 is a cross-sectional view showing a configuration of a modification of the first example of the second embodiment.

FIG. 22 is a perspective view showing a configuration of a modification of the first example of the second embodiment.

FIG. 23 is a perspective view showing a configuration of a first example of a third embodiment.

FIG. 24 is a cross-sectional view taken along line B-B in FIG. 23.

FIG. 25 is a cross-sectional view showing a configuration of a second example of the third embodiment.

FIG. 26 is a cross-sectional view showing a configuration of a third example of the third embodiment.

FIG. 27 is a cross-sectional view showing a configuration of a modification of the first example of the third embodiment.

FIG. 28 is a perspective view showing a configuration of a first example of a fourth embodiment.

FIG. 29 is a perspective view showing a configuration of a second example of the fourth embodiment.

FIG. 30 is a perspective view showing a configuration of a first example of a fifth embodiment.

FIG. 31 is a perspective view showing a configuration of a second example of the fifth embodiment.

FIG. 32 is a perspective view showing an example of a configuration of a sixth embodiment.

### BEST MODE FOR CARRYING OUT THE INVENTION

The present invention is not limited to the following embodiments, and can be appropriately changed, for example, by arbitrarily combining the configurations described as the embodiments and the modifications. In addition, the drawings referred to below are also examples, and the configuration may be simplified or schematically illustrated in the drawings.

### Example of Basic Configuration and Operation of Automatic Hair Transplant Device

First, an example of a basic configuration and operation of an automatic hair transplant device according to an embodiment of the present invention will be described, but for example, a configuration and operation of a known automatic hair transplant device described in JP6533350B1 or the like may be adopted.

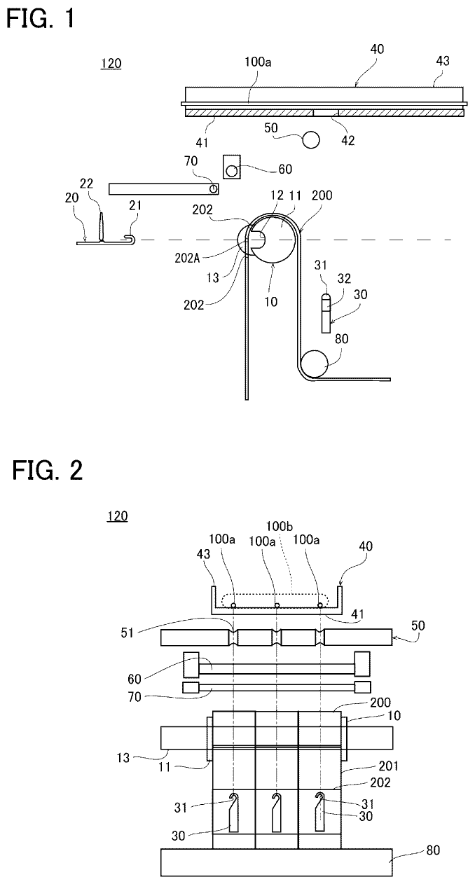

FIG. 1 is a side view of the automatic hair transplant device, and FIG. 2 is a front view of the automatic hair transplant device. As shown in FIGS. 1 and 2, an automatic hair transplant device 120 includes an eccentric 10, a hair transplantation hook needle 20, a pulling hook needle 30, a storage container 40, a yarn positioning bar 50, a yarn dropping bar 60, a yarn pull-in bar 70, a reversing roller 80, and a magnet, a driving mechanism and a control device (not illustrated), and the like. Note that in the side views and the front view shown in FIGS. 1 to 4, the left-right direction in the drawings is the horizontal direction, and the up-down direction is the vertical direction.

A hair material 100a is artificial hair (fiber, synthetic fiber), human hair (hair collected from a human), animal hair (hair collected from an animal other than a human), a mixture thereof, or the like, and includes any hair or hair-like material that can be transplanted by the automatic hair transplant device 120. Further, a hair transplant net 200 has, for example, a mesh shape including a plurality of warp threads 201 and a plurality of weft threads 202.

As shown in FIGS. 1 and 2, a hair material bundle 100b including a plurality of hair materials 100a cut and aligned in advance is arranged in the storage container 40. Hereinafter, the direction of the horizontal plane where the pulling hook needle 30 is present as viewed from the eccentric 10 is referred to as rear, the opposite direction is referred to as front, and these directions are collectively referred to as the front-rear direction. In addition, a direction perpendicular to the front-rear direction on the horizontal plane is referred to as a depth direction, a direction on the left side when viewed from the front to the rear is referred to as back side (back side of the paper in FIG. 1), and a direction on the right side is referred to as front side (front side of the paper in FIG. 1).

The hair transplant net 200 is set so as to cover a net hanging portion 11 of the eccentric 10 from above, and is also in contact with the reversing roller 80 to regulate the position thereof. The storage container 40 has a bottom 41 and a pair of opposed side plates 43, in which the hair material bundle 100b is arranged on the bottom 41. Note that only one storage container 40 may be provided as shown in FIG. 2, or a plurality of storage containers may be provided. Also, the hair materials 100a arranged in the storage container 40 having different types such as color, thickness, and hardness (elasticity) may be mixed with each other.

The hair transplantation hook needle 20 includes a hook 21 at the tip and a latch 22 that opens and closes a needle opening of the hook 21. The hair transplantation hook needle 20 is driven by the driving mechanism controlled by the control device, and ties the hair material 100a to the hair transplant net 200 to transplant the hair material. The pulling hook needle 30 is a member for catching the hair material 100a from the storage container 40, and is provided with a hook 31 capable of catching one hair material (or a plurality of hair materials) 100a at the tip.

The yarn positioning bar 50 is a member for guiding the hair material 100a pulled out from the storage container 40 to a predetermined position by the pulling hook needle 30. The yarn dropping bar 60 vibrates in the up-down direction by the driving mechanism controlled by the control device, and shakes off the hair material 100a pulled in by the yarn pull-in bar 70. The yarn pull-in bar 70 is moved in the front-rear direction by the driving mechanism controlled by the control device, and hooks the hair material 100a transplanted into the hair transplant net 200 and pulls the hair material 100a forward.

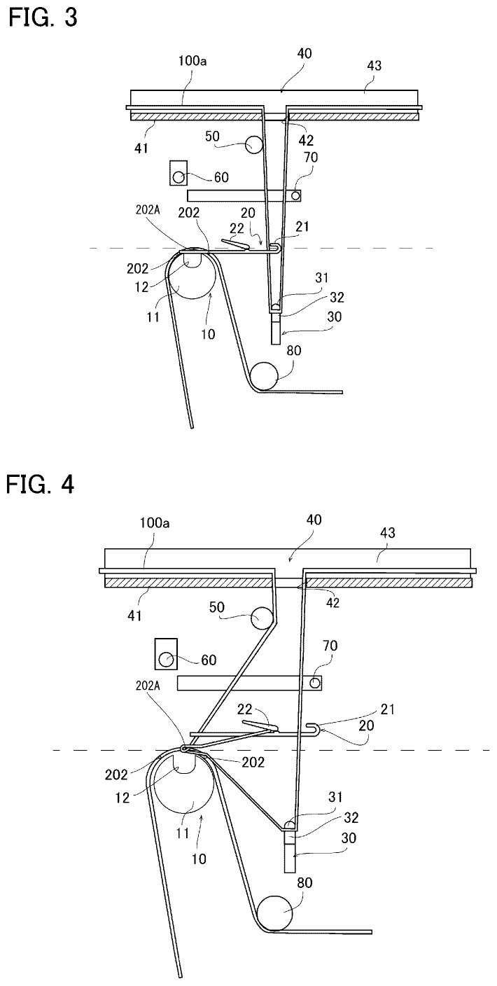

Next, an operation of the automatic hair transplant device will be described. The automatic hair transplant device operates as follows with the state shown in FIG. 1 as an initial state. (1) The yarn pull-in bar 70 advances rearward to a position beyond an outlet 42 of the storage container 40. (2) The pulling hook needle 30 ascends until a distal end including the hook 31 enters the inside of the storage container 40 from the outlet 42, catches the hair material 100a, and descends. (3) The hair transplantation hook needle 20 advances rearward and the hook 21 passes under a weft thread 202-1. (4) A shaft portion 13 is rotated by 90 degrees such that an escape portion 12 of the eccentric 10 faces upward. (5) As shown in FIG. 3, the hair transplantation hook needle 20 advances rearward. (6) The hair transplantation hook needle 20 rotates from a state in which the needle opening of the hook 21 faces upward to a state in which the needle opening faces the hair material 100a (a state in which the needle opening faces the back side) to catch the hair material 100a with the needle opening. (7) The hair transplantation hook needle 20 rotates so that the needle opening of the hook 21 faces upward, and then advances forward and passes below the weft thread 202-1. At this time, the latch 22 comes into contact with the weft thread 202-1 to close the needle opening, but thereafter, the latch 22 is attracted by a magnet (not illustrated) to open the needle opening. (8) The hair transplantation hook needle 20 ascends as it advances backward and rotates to twist the hair material 100a to form a loop. (9) As shown in FIG. 4, the hair transplantation hook needle 20 advances rearward. (10) The hair transplantation hook needle 20 rotates from a state in which the needle opening of the hook 21 faces upward to a state in which the needle opening faces the hair material 100a (a state in which the needle opening faces the back side) to catch the hair material 100a with the needle opening. (11) The hair transplantation hook needle 20 rotates so that the needle opening of the hook 21 faces upward, and then advances forward and passes through the loop of the hair material 100a. At this time, the latch 22 comes into contact with the loop of the hair material 100a to close the needle opening. (12) The yarn pull-in bar 70 advances forward to pull the hair material 100a forward, and returns to the initial state. The eccentric 10 and the hair transplantation hook needle 20 return to the initial state. At this time, the latch 22 is attracted by a magnet (not illustrated) to open the needle opening. The yarn dropping bar 60 moves downward to fix the hair material 100a downward, and then moves upward to return to the initial state.

In the operation (2), after the distal end of the pulling hook needle 30 is inserted into the outlet 42, the distal end may slightly move to the front side (the side where the needle opening 32 of the hook 31 is opened) so as to easily catch the hair material 100a by the hook 31. In the operations (6) and (10), in order to facilitate introduction of the hair material 100a into the needle opening of the hook 21, the pulling hook needle 30 may be moved to the front side, or both the pulling hook needle 30 and the hair transplantation hook needle 20 may be moved so as to approach each other. In FIGS. 3 and 4, the hair transplantation hook needle 20 catches the front side of the hair material 100a folded in two in the operation (6) and catches the rear side in the operation (10), but either the front side or the rear side may be caught in each operation. In the operation of (7), the hair transplantation hook needle 20 may not ascend. Also, in the operation, the hair transplantation hook needle may rotate one or more turns, or may rotate half a turn. In the operation of (11), when the hair transplantation hook needle 20 passes through the loop of the hair material 100a, the pulling hook needle 30 may wiggle up and down. As a result, the hook 31 is detached from the hair material 100a, and peeling in which the hair material 100a is shrunk by friction is prevented.

### Configuration and Operation for Suppressing Defective Take Out of Hair Material

Hereinafter, a description of a specific configuration for suppressing defective take out of the hair material 100a from the storage container 40 will be given.

### First Embodiment

First, a first embodiment of the present invention will be described. The first embodiment includes an energizing part that applies a force including a component in a direction away from the outlet 42 to the hair materials 100a, thereby alleviating the reduction in density of the hair materials 100a in the vicinity of the outlet 42 in the storage container 40. Further, as described above, the hair materials 100a are arranged on the bottom 41 across the outlet 42.

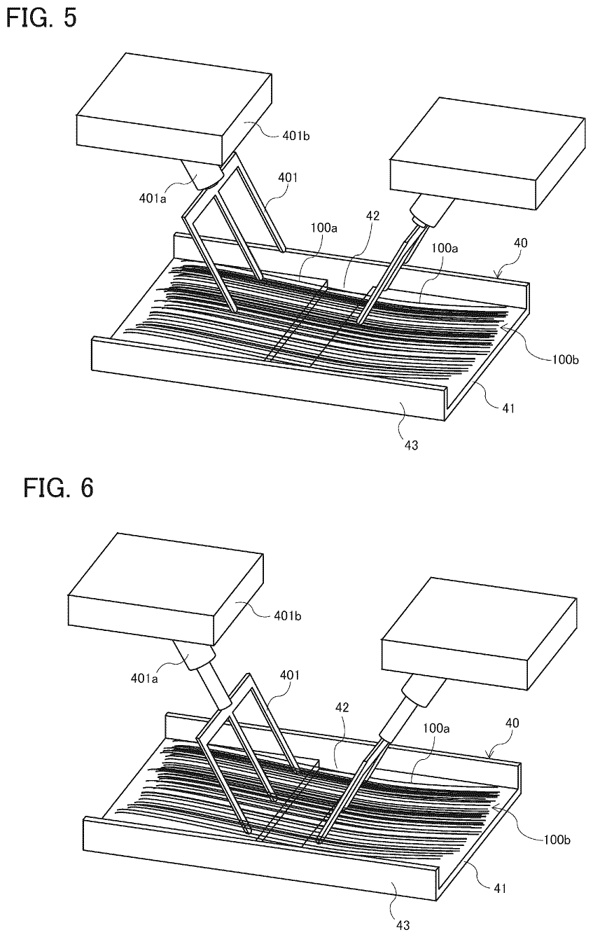

First, a first example of the first embodiment will be described with reference to the drawings. FIGS. 5 to 8 are perspective views showing a configuration of the first example of the first embodiment. Note that FIGS. 5 to 8 show the movement of protrusions 401 as energizing parts in chronological order, and the protrusions 401 move in the order of FIGS. 5, 6, 7, and 8.

As shown in FIG. 5, in the present example, the protrusions 401 are provided as the energizing parts. The protrusions 401 shown in FIG. 5 have a shape like comb teeth, and a plurality of protrusions 401 (three protrusions in this example) are connected to each other. The protrusions 401 are inclined so as to be away from the outlet 42 as the protrusions 401 are farther from the bottom 41. Also, in the present example, the protrusions 401 are provided so as to face each other with the outlet 42 interposed therebetween.

Next, as shown in FIG. 6, the protrusions 401 go straight along the inclination direction, and tips of the protrusions 401 are inserted into the hair material bundle. At this time, the protrusions 401 go straight when being driven by a first reciprocating mechanism 401a such as an air cylinder. In addition, since the protrusions 401 go straight along the inclination direction, the inclination does not change before and after going straight. The operation of the first reciprocating mechanism 401a is controlled by, for example, the above control device.

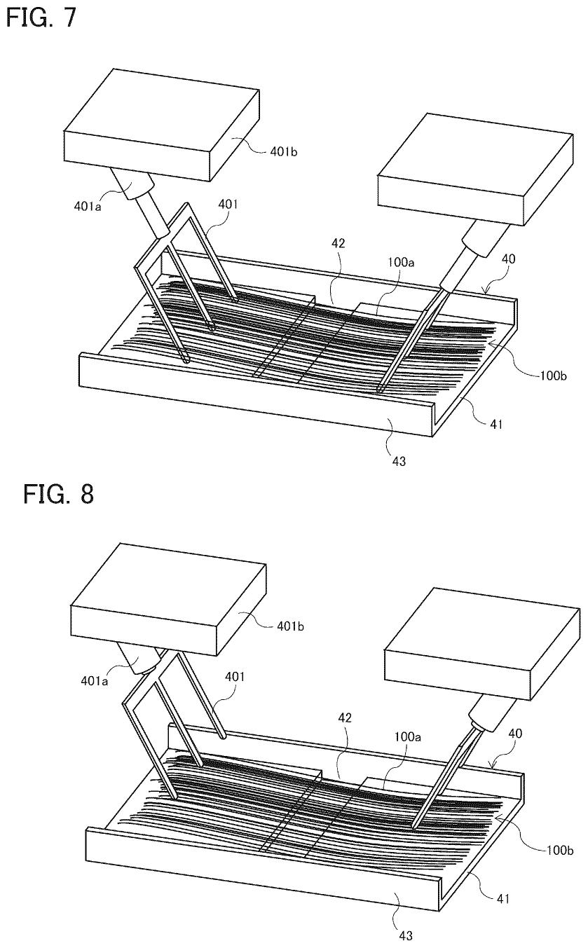

Next, as shown in FIG. 7, the protrusions 401 advance in a direction away from the outlet 42 while maintaining the same inclination as in FIGS. 5 and 6. As a result, since the tips of the protrusions 401 advance along the hair materials 100a, it is possible to apply a force in a direction away from the outlet 42 to the hair materials 100a (a force that alleviates bending and slackening of the hair materials 100a). At this time, the protrusions 401 are driven by a second reciprocating mechanism 401b such as a rack and pinion, for example, to advance in a direction away from the outlet 42. The second reciprocating mechanism 401b drives not only the protrusions 401 but also the first reciprocating mechanism 401a. In addition, the operation of the second reciprocating mechanism 401b is controlled by, for example, the above control device.

Next, as shown in FIG. 8, the protrusions 401 retreat along the inclination direction to be removed from the hair material bundle 100b while maintaining the same inclination as in FIGS. 5 to 7. At this time, the protrusions 401 retreat when being driven by the first reciprocating mechanism 401a.

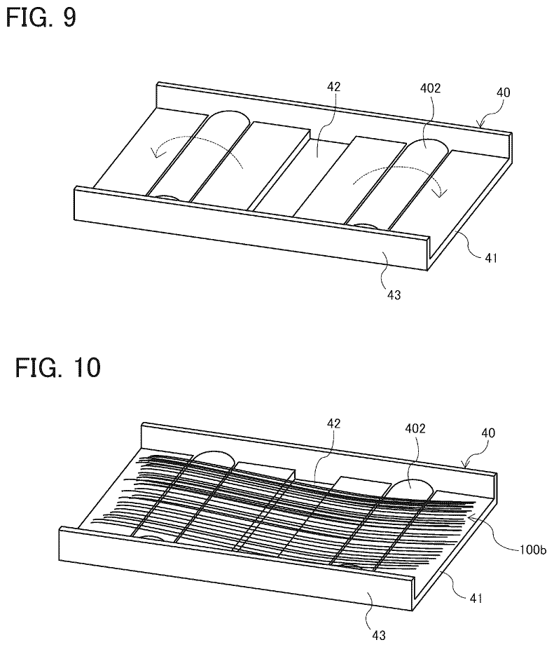

A second example of the first embodiment will be described with reference to the drawings. FIGS. 9 and 10 are perspective views showing a configuration of the second example of the first embodiment. Note that, in FIG. 9, the rotation direction of rollers 402 as energizing parts is shown by a broken line, and in FIG. 10, hair materials 100a are also illustrated in addition to the rollers 402.

As shown in FIGS. 9 and 10, in the present example, the rollers 402 are provided as the energizing parts. Also, the conveying direction of the rollers 402 by rotation is a direction away from the outlet 42. As a result, it is possible to apply a force in a direction away from the outlet 42 to the hair materials 100a (a force that alleviates bending and slackening of the hair materials 100a) by the rotation of the rollers 402.

The rollers 402 are driven by, for example, a motor or the like, and the operation of the motor is controlled by a control device. Furthermore, in the present example, the rollers 402 are provided so as to face each other with the outlet 42 interposed therebetween, and these rollers 402 rotate simultaneously. Although FIGS. 9 and 10 have illustrated the cases where the rollers 402 are provided on the bottom 41 of the storage container 40, the roller 402 may be provided on the side plate 43 or may be provided at a position facing the bottom 41 (above the hair materials 100a).

As described above, when the energizing parts (the protrusions 401, the rollers 402) that apply a force including a component in a direction away from the outlet to the hair materials 100a is provided, the hair materials 100a are pulled in a direction away from the outlet. Then, the bending and slackening are alleviated by stretching the hair materials 100a. As a result, the reduction in density of the hair materials 100a in the vicinity of the outlet 42 in the storage container 40 is improved, and the pulling hook needle 30 can stably catch the hair material at the outlet 42, so that the hair material can be stably transplanted.

In the above examples, the cases where both the protrusions 401 and the rollers 402 are provided so as to face each other with the outlet 42 interposed therebetween have been described, but they may be provided only on one side of the outlet 42. In this case, the entire hair material bundle 100b can move, but the bending and slackening of the hair materials 100a can be alleviated. Furthermore, since the bending and slackening of the hair materials 100a in the vicinity of the outlet 42 move to a position shifted from the outlet 42 as the entire hair material bundle 100b moves, this also improves the reduction in density of the hair materials 100a in the vicinity of the outlet 42 in the storage container 40. Even in a case where the protrusions 401 and the rollers 402 are provided so as to face each other with the outlet 42 interposed therebetween, when driving only one side, the same effect as in a case of providing only one side can be obtained, and in a case where one side and the opposite side are alternately driven, the hair material bundle 100b reciprocates with the outlet 42 interposed therebetween, so that it is possible to suppress the position of the hair material bundle 100b from greatly varying.

On the other hand, as in the above example, when the protrusions 401 and the rollers 402 are provided so as to face each other with the outlet 42 interposed therebetween, and are simultaneously driven, the hair materials 100a are pulled toward both end sides, so that the bending and slackening of the hair materials 100a can be effectively alleviated. Note that it is not always necessary to drive the protrusions 401 and the rollers 402 in synchronization, and this effect can be obtained if there is a timing of driving at the same time.

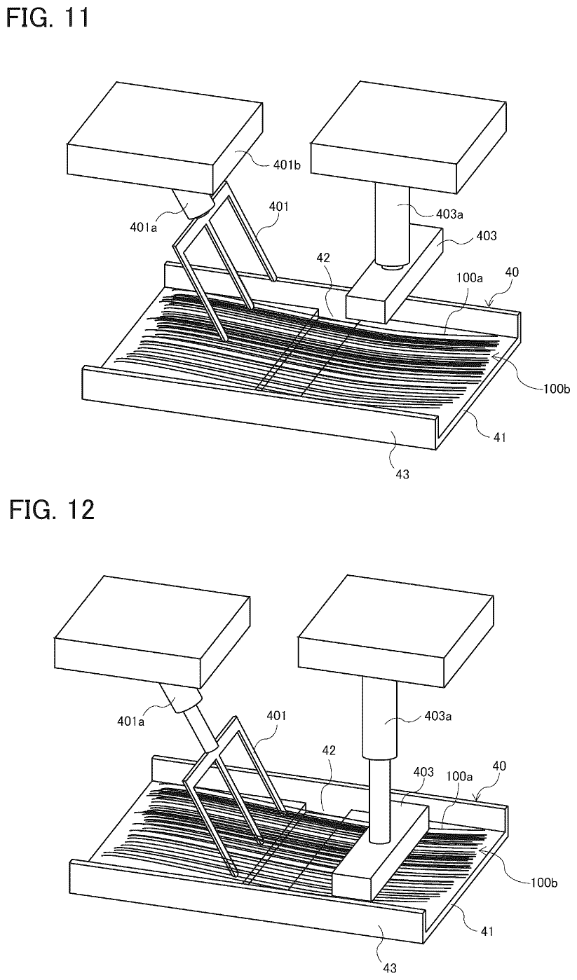

In addition, the mechanisms provided so as to face each other with the outlet 42 interposed therebetween may be different. For example, the protrusions 401 and the roller 402 may be provided so as to face each other with the outlet 42 interposed therebetween. For example, as shown in FIGS. 11 to 14, the protrusions 401 may be provided on one side of the outlet 42, and a restraining part 403 that presses the hair material bundle 100b against the bottom 41 to restrict movement may be provided on the opposite side. FIGS. 11 to 14 correspond to FIGS. 5 to 8 showing the first example described above. As shown in FIGS. 11 to 14, the restraining part 403 presses the hair material bundle 100b against the bottom 41 to restrict the movement of the hair material bundle 100b when the tips of the protrusions 401 are inserted into the hair material bundle 100b (the state shown in FIGS. 12 and 13), that is, when the protrusions 401 can apply a force including a component in a direction away from the outlet 42 to the hair material bundle 100b. Note that the restraining part 403 is driven by a reciprocating mechanism 403a such as an air cylinder, and the operation of the reciprocating mechanism 403a is controlled by, for example, the above control device. Further, although FIGS. 11 to 14 have illustrated the cases where the protrusions 401 and the restraining part 403 are combined, it is also possible to combine the roller 402 and the restraining part 403.

In addition, in the first example, the protrusions 401 may not be inclined and may be perpendicular to the bottom 41. However, when the protrusions 401 are inclined as described above, the protrusions 401 can apply a force having not only a component in a direction away from the outlet 42 but also a component in a downward direction to the hair materials 100a. As a result, since the hair materials 100a in the storage container 40 are pressed toward the bottom 41 side having the outlet 42, it is possible to effectively improve the reduction in density of the hair materials 100a in the vicinity of the outlet 42 in the storage container 40. Also, in a case of a configuration in which the inclination of the protrusions 401 does not change during a series of operations, for example, the protrusions 401 can be caused to perform the operations of FIGS. 5 to 8 by a simple mechanism including only the first reciprocating mechanism 401a and the second reciprocating mechanism 401b.

Further, in the first example, when the tips of the protrusions 401 are inserted into the hair material bundle 100b, then the protrusions 401 advance in a direction away from the outlet 42, and then the tips of the protrusions 401 are removed from the hair material bundle 100b, it is possible to apply only a force that alleviates bending and slackening to the hair materials 100a without applying a force that promotes the bending and slackening (a force having a component in a direction approaching the outlet 42).

Furthermore, in the first example, the plurality of protrusions 401 may be configured to be driven by one mechanism. For example, in a case where a plurality of storage containers 40 is provided, the protrusions 401 provided for each of the plurality of storage containers 40 may be driven by one set of the first reciprocating mechanism 401a and the second reciprocating mechanism 401b, or may be driven by the first reciprocating mechanism 401a provided for each protrusion 401 and one second reciprocating mechanism 401b. Similarly, the plurality of rollers 402 and the plurality of restraining parts 403 may also be configured to be driven by one mechanism.

### Second Embodiment

Next, a second embodiment of the present invention will be described. In the second embodiment, the bottom 41 of the storage container 40 has a depressed portion that is concave in the cross section perpendicular to the direction in which the hair materials 100a straddles the outlet 42 (hereinafter, referred to as the “longitudinal direction”.), thereby alleviating the reduction in density of the hair materials 100a in the vicinity of the outlet 42 in the storage container 40. The longitudinal direction is a direction parallel to the direction away from the outlet 42 described in the first embodiment.

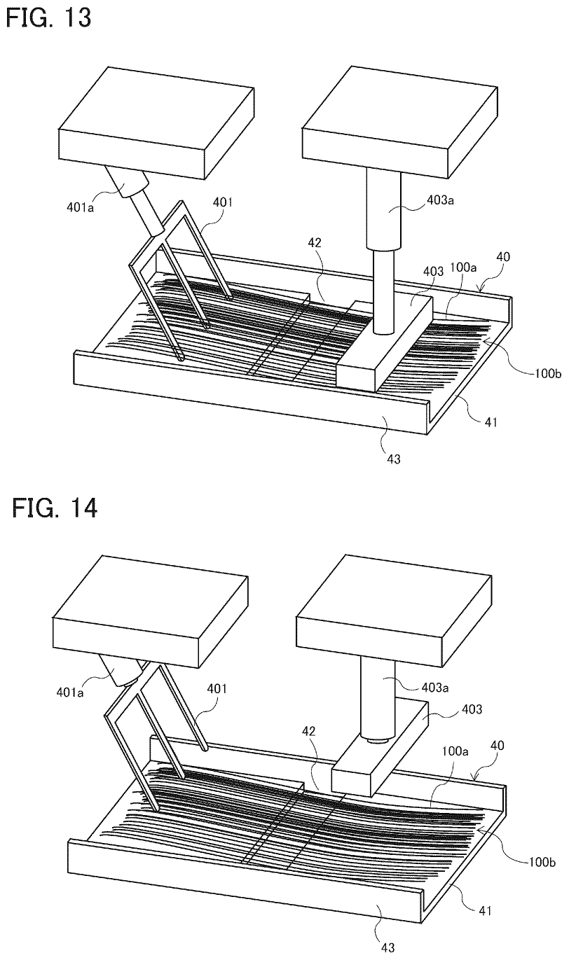

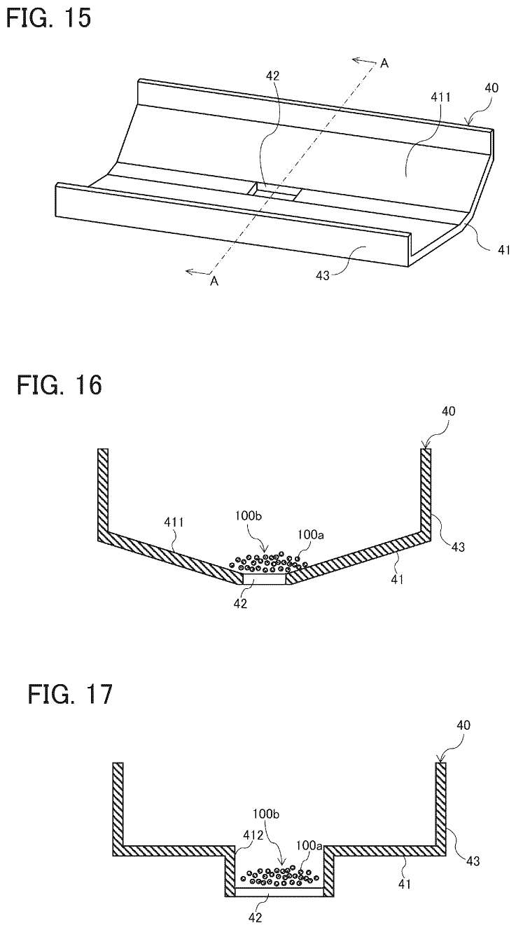

First, a first example of the second embodiment will be described with reference to the drawings. FIG. 15 is a perspective view showing a configuration of the first example of the second embodiment. FIG. 16 is a cross-sectional view taken along line A-A (a cross section perpendicular to the longitudinal direction and passing through the outlet 42) in FIG. 15. In FIG. 15, an illustration of the hair materials 100a is omitted in order to make the configuration of the bottom 41 easily viewable. Also, in the cross-sectional view of FIG. 16, the left-right direction is the horizontal direction, and the up-down direction is the vertical direction.

As shown in FIGS. 15 and 16, in the second embodiment, an upper surface of the bottom 41 of the storage container 40 on which the hair materials 100a are arranged is not flat, and the storage container 40 has a depressed portion 411 that is concave in a cross section perpendicular to the longitudinal direction. Also, in the depressed portion 411, an outlet 42 is formed at a lower end which is a bottom.

In addition, as shown in FIG. 16, the depressed portion 411 of the first example has a width in the horizontal direction in the illustrated cross section that is narrower toward the lower end and is a linear slope. Although FIGS. 15 and 16 have illustrated the cases where the depressed portion 411 is formed on the entire surface of the bottom 41, the depressed portion 411 may be formed only at the outlet 42 and the periphery thereof. In this case, a portion of the bottom 41 where the depressed portion 411 is not provided may be flat.

A second example of the second embodiment will be described with reference to the drawings. FIG. 17 is a cross-sectional view showing a configuration of the second example of the second embodiment. FIG. 17 is a cross-sectional view showing a cross section corresponding to FIG. 16 showing the first example of the second embodiment.

As shown in FIG. 17, similarly to the depressed portion 411 of the first example, a depressed portion 412 of the second example that is concave in the illustrated cross section, and an outlet 42 is formed at a lower end which is a bottom. On the other hand, the depressed portion 412 of the second example has a wall surface parallel to the vertical direction, whose width to the lower end in the horizontal direction in the illustrated cross section is constant.

As described above, when the bottom 41 has the depressed portion 411 or 412 that is concave in a cross section perpendicular to the longitudinal direction, the depressed portion 411 or 412 regulates a movement range of the hair materials 100a to be in the vicinity of the outlet 42. As a result, the reduction in density of the hair materials 100a in the vicinity of the outlet 42 in the storage container 40 is improved, and the pulling hook needle 30 can stably catch the hair material 100a at the outlet 42, so that the hair material can be stably transplanted.

Further, as in the first example, when the width of the depressed portion 411 in the horizontal direction gradually decreases toward the lower end in the cross section shown in FIG. 16, a force along the slope of the depressed portion 411 due to the own weight of the hair materials 100a, that is, a force in a direction toward the outlet 42 can be applied to the hair materials 100a on the depressed portion 411. Furthermore, as in the first example, when the depressed portion 411 has a linear slope in the cross section shown in FIG. 16, a force in a direction toward the outlet 42 is uniformly applied to the hair materials 100a on the depressed portion 411, and the entire hair materials 100a on the depressed portion 411 can be directed toward the outlet 42.

On the other hand, as in the second example, in the cross section shown in FIG. 17, when the width of the depressed portion 412 in the horizontal direction is uniform toward the lower end, it is possible to strongly restrict the hair materials 100a on the depressed portion 412 from moving to the outside of the depressed portion 412.

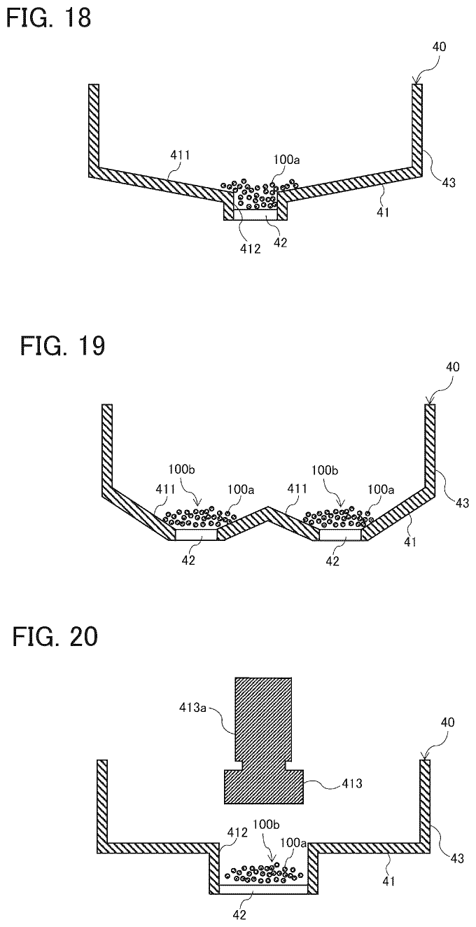

Note that the first example and the second example can be combined. FIG. 18 is a cross-sectional view showing a configuration in which the first example and the second example of the second embodiment are combined. Also, FIG. 18 is a cross-sectional view showing a cross section corresponding to FIG. 16 showing the first example of the second embodiment. In the example shown in FIG. 18, a depressed portion 411 similar to that of the second example is formed in the vicinity of the lower end of a depressed portion 412 similar to that of the first example. In this case, a force in a direction toward the outlet 42 is applied to the hair materials 100a on the depressed portion 411, and outward movement of the hair materials 100a on the depressed portion 412 is strongly regulated.

Further, although the above examples have illustrated the cases where one depressed portion 411 or 412 and one outlet 42 are formed in one storage container 40, a plurality of depressed portions 411 or 412 and outlets 42 may be formed in one storage container 40. FIG. 19 is a cross-sectional view in a case where the storage container 40 includes a plurality of depressed portions 411 of the first example. Also, FIG. 19 is a cross-sectional view showing a cross section corresponding to FIG. 16 showing the first example of the second embodiment. As shown in FIG. 19, when the storage container 40 includes the plurality of depressed portions 411, it is possible to improve the reduction in density of the hair materials 100a in the vicinity of the outlet 42 of each of the depressed portions 411 even when the plurality of hair materials 100a are simultaneously taken out.

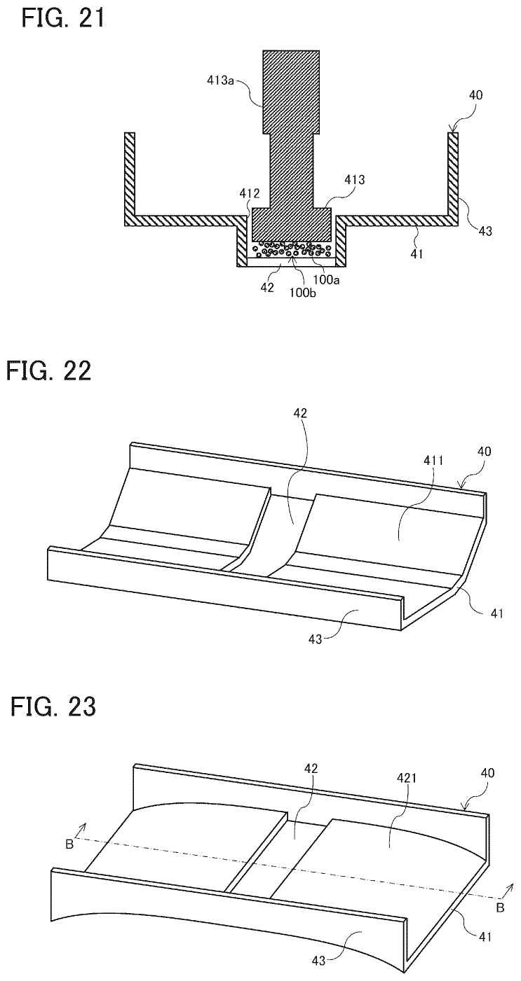

Further, not only the depressed portion 411 or 412 is provided, but also a pressing part that presses the hair materials 100a and the hair material bundle 100b downward may be provided. FIGS. 20 and 21 are cross-sectional views showing a configuration further including a pressing part 413 in a configuration of the second example of the second embodiment. Also, FIGS. 20 and 21 are cross-sectional views showing a cross section corresponding to FIG. 16 showing the first example of the second embodiment. As shown in FIGS. 20 and 21, the pressing part 413 has a shape fitted into the depressed portion 412, and is pushed downward to press the hair materials 100a (hair material bundle 100b) downward as shown in FIG. 21. As a result, the pressing part 413 can apply a force toward the outlet 42 to the hair materials 100a on the depressed portion 412. Note that the pressing part 413 is driven by, for example, a reciprocating mechanism 413a such as an air cylinder, and the operation of the reciprocating mechanism 413a is controlled by, for example, the above control device.

Further, although FIGS. 15 to 21 have illustrated the cases where the outlet 42 is provided only in a part of the bottom 41 in the horizontal direction perpendicular to the longitudinal direction, similarly to the bottom 41 illustrated in FIGS. 5 to 14, the outlet 42 may be formed from one side plate 43 to the other side plate 43 of the bottom 41. FIG. 22 is a perspective view showing a case where the outlet 42 is formed from one side plate 43 to the other side plate 43 of the bottom 41 in the configuration of the first example of the second embodiment. In the example shown in FIG. 22, the outlet 42 is formed not only on the lower end of the depressed portion 411 but also on a linear slope. Even with such a configuration, similarly to the above example, the depressed portion 411 regulates the movement range of the hair materials 100a to be in the vicinity of the outlet 42, so that it is possible to improve the reduction in density of the hair materials 100a in the vicinity of the outlet 42 in the storage container 40.

Further, the above examples have illustrated, for example, as shown in FIGS. 15 and 22, the cases where the depressed portion 411 is formed from one end to the other end in the longitudinal direction of the bottom 41. In this case, the movement range of the hair materials 100a can be regulated so as to be in the vicinity of the outlet 42 over the entire length in the longitudinal direction. On the other hand, the depressed portion 411 or 412 may be formed only in a part of the bottom 41 in the longitudinal direction. However, in this case, the depressed portion 411 or 412 is formed such that at least the outlet 42 is included in the depressed portion 411 or 412.

### Third Embodiment

Next, a third embodiment of the present invention will be described. In the third embodiment, the bottom 41 of the storage container 40 has a raised portion that is convex in the vertical cross section parallel to the longitudinal direction, thereby alleviating the reduction in density of the hair materials 100a in the vicinity of the outlet 42 in the storage container 40.

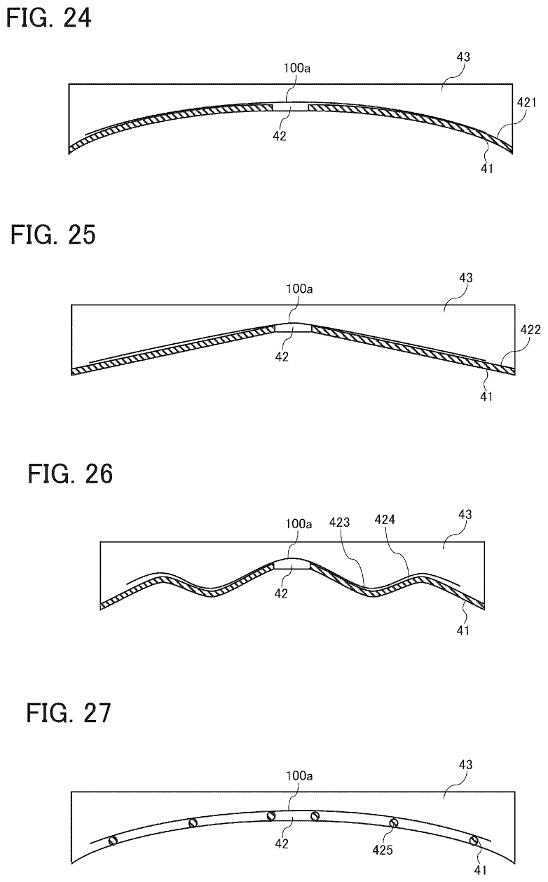

First, a first example of the third embodiment will be described with reference to the drawings. FIG. 23 is a perspective view showing a configuration of the first example of the third embodiment. FIG. 24 is a cross-sectional view taken along line B-B (a vertical cross section parallel to the longitudinal direction) in FIG. 23. In FIG. 23, an illustration of the hair materials 100a is omitted in order to make the configuration of the bottom 41 easily viewable. Also, in the cross-sectional view of FIG. 24, the left-right direction is the horizontal direction, and the up-down direction is the vertical direction.

As shown in FIGS. 23 and 24, in the third embodiment, an upper surface of the bottom 41 of the storage container 40 on which the hair materials 100a are arranged is not flat, and the storage container 40 has a raised portion 421 that is convex in a vertical cross section parallel to the longitudinal direction of the hair materials 100a. Also, in the raised portion 421, an outlet 42 is formed at an upper end which is a top.

In addition, as shown in FIG. 24, the raised portion 421 of the first example has a shape in which the inclination with respect to the horizontal direction increases as it goes away from the outlet 42 in the illustrated cross section. Although FIGS. 23 and 24 have illustrated the cases where the raised portion 421 is formed on the entire surface of the bottom 41, the raised portion 421 may be formed only at the outlet 42 and the periphery thereof. In this case, a portion of the bottom 41 where the raised portion 421 is not provided may be flat.

A second example of the third embodiment will be described with reference to the drawings. FIG. 25 is a cross-sectional view showing a configuration of the second example of the third embodiment. FIG. 25 is a cross-sectional view showing a cross section corresponding to FIG. 24 showing the first example of the third embodiment.

As shown in FIG. 25, similarly to the raised portion 421 of the first example, a raised portion 422 of the second example has a shape protruding upward with the outlet 42 as an upper end in the illustrated cross section. On the other hand, the raised portion 422 of the second example has a constant inclination with respect to the horizontal direction in the illustrated cross section, and has a linear shape. Although FIG. 25 has illustrated the case where the raised portion 422 is formed on the entire surface of the bottom 41, the raised portion 422 may be formed only at the outlet 42 and the periphery thereof. In this case, a portion of the bottom 41 where the raised portion 422 is not provided may be flat.

A third example of the third embodiment will be described with reference to the drawings. FIG. 26 is a cross-sectional view showing a configuration of the third example of the third embodiment. FIG. 26 is a cross-sectional view showing a cross section corresponding to FIG. 24 showing the first example of the third embodiment.

As shown in FIG. 26, similarly to the raised portions 421, 422 of the first example and the second example, a raised portion 423 of the third example is convex in the illustrated cross section. On the other hand, in the raised portion 423 of the third example, the raised portion 423 is formed only at the outlet 42 and the periphery thereof, and an inclined portion 424 inclined so as to be upper as it goes away from the outlet 42 is provided on the outer side thereof. Although FIG. 26 has illustrated the case where the outer side of the inclined portion 424 is inclined so as to be lower as it goes away from the outlet 42 similarly to the raised portion 423, the outer side of the inclined portion 424 may be flat, or the inclined portion 424 may continue to the end in the longitudinal direction of the bottom 41.

As described above, when the bottom 41 has any of the raised portions 421 to 423 that are convex in the vertical cross section parallel to the longitudinal direction, the hair materials 100a are supported in a convex shape along any of the raised portions 421 to 423, whereby the hair materials 100a are pulled along the longitudinal direction by their own weight, so that the bending and slackening of the hair materials 100a are suppressed, and the reduction in density of the hair materials 100a in the vicinity of the outlet 42 in the storage container 40 is improved. As a result, the pulling hook needle 30 can stably catch the hair material 100a at the outlet 42, so that the hair material can be stably transplanted.

Further, as in the first example, when the raised portion 421 is formed into a shape in which the inclination with respect to the horizontal direction increases as it goes away from the outlet 42 in the cross section shown in FIG. 24, the hair materials 100a are curved along the raised portion 421 and hardly float, and the friction between the hair materials 100a and the raised portion 421 increases, so that the movement of the hair materials 100a are restricted. Therefore, it is possible to effectively improve the reduction in density of the hair materials 100a in the vicinity of the outlet 42 in the storage container 40.

On the other hand, as in the second example, when the raised portion 422 has a shape in which the inclination with respect to the horizontal direction is constant in the cross section (that is, a linear shape) shown in FIG. 25, the hair materials 100a are linearly pulled by their own weight, so that the bending and slackening of the hair materials 100a are effectively suppressed. Therefore, it is possible to effectively improve the reduction in density of the hair materials 100a in the vicinity of the outlet 42 in the storage container 40.

Further, as in the third example, in the cross section shown in FIG. 26, when an inclined portion inclined so as to be upper as it goes away from the outlet 42 is provided on the outer side of the raised portion 423, the hair materials 100a are curved and supported a plurality of times, so that the length in the longitudinal direction of the storage container 40 that houses the hair materials 100a can be shortened.

Note that the bottom 41 and the raised portion may not be continuous surfaces as long as the hair materials 100a are only supported in a convex shape. FIG. 27 is a cross-sectional view showing a configuration of a modification of the first example of the third embodiment. Also, FIG. 27 is a cross-sectional view showing a cross section corresponding to FIG. 24 showing the first example of the third embodiment. In the example shown in FIG. 27, a raised portion 425 includes a plurality of rod-shaped members perpendicular to the longitudinal direction and extending in the horizontal direction, and a gap is formed between the adjacent rod-shaped members. Even in the case shown in FIG. 27, the hair materials 100a are supported in a convex shape by the raised portion 425.

On the other hand, as in the first example to the third example described above, when the bottom 41 and any of the raised portions 421 to 423 are a surface continuous from the outlet 42 to one end in the longitudinal direction in the vertical cross section parallel to the longitudinal direction, it is possible to prevent a situation in which the hair material 100a is pulled out downward from a portion other than the outlet 42 and is entangled in the driving mechanism of the automatic hair transplant device 120 from occurring. Here, the continuous surface means a surface continuing without a gap, and a surface including a steeply bending point (non-differentiable point) is also included in the continuous surface.

Also, in the third embodiment, a force that pulls both ends of the hair materials 100a along the longitudinal direction may be applied to the hair materials 100a. For example, in the case of using the storage container 40 having the raised portion 421 or 422 of the first example or the second example, both ends of the hair material bundle 100b may be bundled with an elastic body such as a rubber band, and then a weight may be added to the elastic body. As a result, a pulling force in the longitudinal direction can be applied to the hair materials 100a more than their own weight, so that the bending and slackening of the hair materials 100a can be more effectively suppressed.

Further, in the third embodiment, the raised portion may have a wall surface parallel to the vertical direction. However, in this case, the hair materials 100a are less likely to come into contact with the wall surface, and the friction between the hair materials 100a and the raised portion is reduced.

### Fourth Embodiment

Next, a fourth embodiment of the present invention will be described. The fourth embodiment includes a hooking position changing part that changes the position at which the pulling hook needle 30 hooks with respect to the hair material 100a arranged in the storage container 40, thereby alleviating the reduction in density of the hair materials 100a in the vicinity of the outlet 42 in the storage container 40.

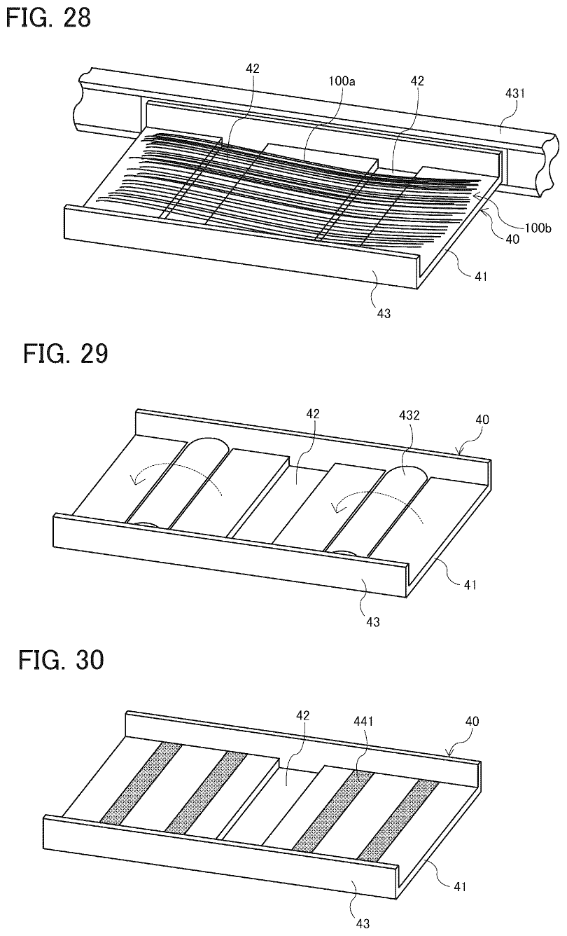

First, a first example of the fourth embodiment will be described with reference to the drawing. FIG. 28 is a perspective view showing a configuration of the first example of the fourth embodiment. As shown in FIG. 28, in the first example of the fourth embodiment, the storage container 40 has a plurality of (two in the illustrated example) outlets 42 at the bottom 41. The outlets 42 are arranged along the longitudinal direction, and the hair materials 100a are arranged on the bottom 41 so as to straddle the plurality of outlets 42.

In addition, as shown in FIG. 28, in the first example of the fourth embodiment, a storage container driving part 431 that drives the storage container along the longitudinal direction is provided as the hooking position changing part. The storage container driving part 431 includes, for example, a reciprocating mechanism such as a rack and pinion, and the operation is controlled by, for example, the above control device. When the storage container 40 is driven along the longitudinal direction by the storage container driving part 431, the outlet 42 into which the pulling hook needle 30 is inserted next is changed. Note that the pulling hook needle 30 does not need to be driven in the longitudinal direction, and only needs to be driven in the vertical direction as described above.

A second example of the fourth embodiment will be described with reference to the drawing. FIG. 29 is a perspective view showing a configuration of the second example of the fourth embodiment. In FIG. 29, the rotation direction of the roller is shown by a broken line, and an illustration of the hair materials 100a is omitted, but when the hair materials 100a are illustrated, the rotation direction is similar to that in FIG. 10 shown for the first embodiment.

As shown in FIG. 29, in the second example of the fourth embodiment, a hair material driving part 432 that moves the hair materials 100a arranged in the storage container 40 along the longitudinal direction (more specifically, a roller which conveys the hair materials 100a and the hair material bundle 100b) is provided as the hooking position changing part. Similarly to the first embodiment shown in FIG. 9, two rollers as the hair material driving part 432 are provided along the longitudinal direction, but unlike in FIG. 9, the rotation direction (conveying direction) of the rollers is the same. The roller is driven by, for example, a motor, and the operation of the motor is controlled by, for example, the above control device. In addition, these rollers rotate simultaneously, and the entire hair materials 100a and the entire hair material bundle 100b move to the outlet 42 of the storage container 40. Although FIG. 29 illustrates a case where two rollers are provided, only one roller may be provided, or three or more rollers may be provided.

As described above, with the hooking position changing part 431 or 432 that changes the position at which the pulling hook needle 30 hooks with respect to the hair material 100a arranged in the storage container 40, even if the hair material 100a are bent or slackened by taking out the hair material 100a by the pulling hook needle 30, the hair material 100a can be taken out by hooking the pulling hook needle 30 on another place where the bending and slackening do not occur. As a result, the pulling hook needle 30 can stably catch the hair material 100a at the outlet 42, so that the hair material can be stably transplanted.

Furthermore, as in the first example and the second example, by providing the storage container driving part 431 and the hair material driving part 432 as the hooking position changing part, it is possible to change the position at which the pulling hook needle 30 hooks with respect to the hair material 100a without requiring the driving of the pulling hook needle 30 related to the hair transplantation operation. In addition, when the storage container driving part 431 is provided as in the first example, it is possible to loosen the hair materials 100a to alleviate the bending and slackening by driving the entire storage container 40. On the other hand, when the hair material driving part 432 is provided as in the second example, it is possible to loosen the hair materials 100a to alleviate the bending and slackening by driving the hair materials 100a. Also, in the case of the second example, since a plurality of portions of the hair materials 100a positioned at one outlet 42 can be selected by controlling the rotation amount of the roller, it is possible to make the pulling hook needle 30 hook on many portions where no bending or slackening occurs.

Note that, in the configuration of the second example shown in FIG. 29, a plurality of outlets 42 may be provided in the bottom 41 of the storage container 40 as in the first example. Also, the first example and the second example can be combined, and the storage container driving part 431 and the hair material driving part 432 may be provided at the same time.

Further, the hooking position changing part may change the position at which the pulling hook needle 30 hooks with respect to the hair material 100a by driving the pulling hook needle 30. However, in this case, for example, it is necessary to perform two-stage driving in which the pulling hook needle 30 is driven in the vertical direction to approach the bottom of the storage container 40 and then driven in the longitudinal direction.

Furthermore, in FIG. 29 showing the second example, a case where the rollers rotate in one direction is shown, but the rollers may rotate in the opposite direction. In this case, when the rollers are rotated in one direction to take out the hair material 100a with the pulling hook needle 30, and then the rollers are rotated in the opposite direction by a different rotation amount, the position at which the pulling hook needle 30 hooks with respect to the hair material 100a is different. In addition, even if the rotation amount of the roller is the same, the hair materials 100a are loosened by conveying the hair materials 100a with the roller, and the bending and slackening are alleviated by hooking the pulling hook needle 30 on a portion different from the portion where the bending and slackening of the hair materials 100a have occurred. Therefore, the pulling hook needle 30 can hook on the portion where the bending and slackening of the hair materials 100a have been alleviated.

### Fifth Embodiment

Next, a fifth embodiment of the present invention will be described. In the fifth embodiment, an anti-slip portion that suppresses slippage of the hair materials 100a more than the smooth surface of the material constituting the storage container 40 is provided at a portion of the storage container 40 in contact with the hair materials 100a, thereby alleviating the reduction in density of the hair materials 100a in the vicinity of the outlet 42 in the storage container 40. Note that the smooth surface of the material constituting the storage container 40 is typically a portion of the storage container 40 where the anti-slip portion is not formed, but for example, in a case where the entire surface of the storage container 40 is processed to be a uniform surface, a surface of an unprocessed plate material used for manufacturing the storage container 40 or a surface from which the anti-slip portion is removed corresponds thereto.

First, a first example of the fifth embodiment will be described with reference to the drawings. FIG. 30 is a perspective view showing a configuration of the first example of the fifth embodiment. In FIG. 30, an illustration of the hair materials 100a is omitted in order to make the configuration in the storage container 40 easily viewable.

As shown in FIG. 30, in the first example of the fifth embodiment, an adhesive material 441 is provided as an anti-slip portion on the upper surface of the bottom 41 on which the hair materials 100a are arranged. As the adhesive material 441, for example, a well-known adhesive material such as a rubber-based adhesive material, an acrylic adhesive material, a silicone-based adhesive material, or a urethane-based adhesive material can be applied. Although FIG. 30 has illustrated the case where the adhesive material 441 is provided at two locations on both sides in the longitudinal direction around the outlet 42, the adhesive material 441 may be provided on the entire upper surface of the bottom 41, or the adhesive material 441 may be provided on the side plate 43.

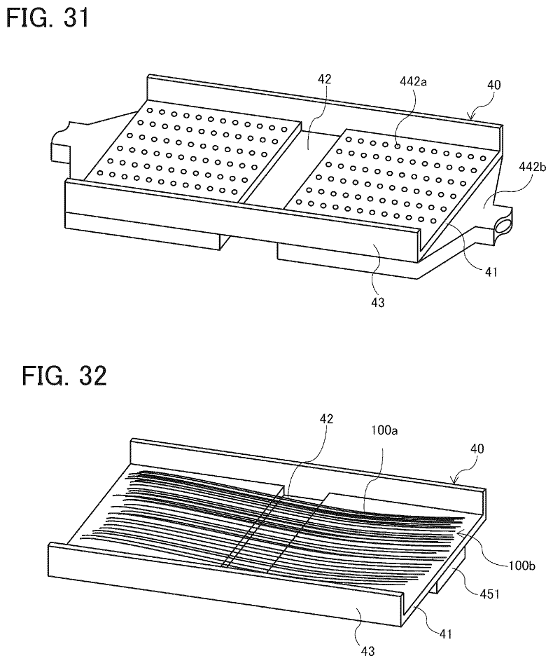

A second example of the fifth embodiment will be described with reference to the drawings. FIG. 31 is a perspective view showing a configuration of the second example of the fifth embodiment. In FIG. 31, an illustration of the hair materials 100a is omitted in order to make the configuration in the storage container 40 easily viewable.

As shown in FIG. 31, in the second example of the fifth embodiment, exhaust holes 442a are provided for adsorbing the hair materials 100a by exhaust as anti-slip portions on the upper surface of the bottom 41 on which the hair materials 100a are arranged. In addition, an exhaust part 442b that exhausts air from the exhaust holes 442a is provided below the bottom 41, and a blower, a compressor, or the like for exhaust is provided ahead of the exhaust part 442b, and the operation of the blower or the compressor, or a valve that switches the presence or absence of exhaust provided in the exhaust part 442b is controlled by, for example, the above control device. Although FIG. 31 has illustrated the case where the exhaust holes 442a are provided in the entire upper surface of the bottom 41, the exhaust holes 442a may be provided only in a part of the upper surface of the bottom 41, or the exhaust holes 442a may be provided in the side plate 43.

As described above, when the anti-slip portion 441 or 442a is provided at the portion of the storage container 40 in contact with the hair materials 100a, the movement of the hair materials 100a is suppressed by the anti-slip portion 441 or 442a, so that the reduction in density of the hair materials 100a in the vicinity of the outlet 42 in the storage container 40 is improved. As a result, the pulling hook needle 30 can stably catch the hair material 100a at the outlet 42, so that the hair material can be stably transplanted.

In addition, as in the first example and the second example, when the anti-slip portion 441 or 442a is provided on at least a part of the upper surface of the bottom 41 on which the hair materials 100a are arranged, the movement of the hair materials 100a is suppressed on the upper surface on which the outlet 42 is provided, so that it is possible to effectively improve the reduction in density of the hair materials 100a in the vicinity of the outlet 42 in the storage container 40.

Also, when the adhesive material 441 is provided as the anti-slip portion as in the first example, with a simple configuration in which the adhesive material 441 is installed in the storage container 40, it is possible to improve the reduction in density of the hair materials in the vicinity of the outlet 42 in the storage container 40, and it is possible to suppress the movement of the hair materials 100a without damaging the hair materials 100a since the hair materials 100a are not rubbed against any object.

On the other hand, when the exhaust holes 442a are provided as the anti-slip portions as in the first example, it is possible to improve the reduction in density of the hair materials 100a in the vicinity of the outlet 42 in the storage container 40 semi-permanently, and it is possible to release the adsorption of the hair materials 100a by stopping the exhaust, so that the hair materials 100a can be easily taken in and out of the storage container 40. In order to prevent the hair materials 100a from being sucked, the exhaust holes 442a are preferably smaller than the outlet 42, for example.

Note that the first example and the second example can be combined. For example, the adhesive material 441 may be provided on a part of the upper surface of the bottom 41, and the exhaust holes 442a may be provided in the other part. Further, the anti-slip portion may be provided by forming unevenness on the bottom 41 and the side plate 43 by, for example, blast treatment or the like.

### Sixth Embodiment

Next, a sixth embodiment of the present invention will be described. The sixth embodiment includes a vibration unit that applies vibration to the storage container 40, thereby alleviating the reduction in density of the hair materials 100a in the vicinity of the outlet 42 in the storage container 40.

The sixth embodiment will be described with reference to the drawing. FIG. 32 is a perspective view showing an example of a configuration of the sixth embodiment. In the configuration example shown in FIG. 32, a vibration unit 451 that applies vibration to the storage container 40 is provided at the bottom 41. The vibration unit 451 includes, for example, an eccentric motor, a solenoid, a piezo element, and the like, and the operation is controlled by, for example, the above control device.

In the configuration example shown in FIG. 32, when the vibration unit 451 generates vibration, the storage container 40 vibrates, and the vibration is propagated to the hair materials 100a. Furthermore, the vibration unit 451 applies a vibration of a frequency of, for example, 1 Hz or more to the storage container 40.

As described above, since the hair materials 100a are loosened by the vibration applied to the storage container 40 by the vibration unit 451, the bending and slackening of the hair materials 100a are alleviated, and the reduction in density of the hair materials 100a in the vicinity of the outlet 42 in the storage container 40 is improved. As a result, the pulling hook needle 30 can stably catch the hair material 100a at the outlet 42, so that the hair material 100a can be stably transplanted.

In particular, the vibration unit 451 applies continuous vibration to the storage container 40 instead of a pulse impact, so that the hair materials 100a can be effectively loosened, and the bending and slackening of the hair materials 100a can be effectively alleviated.

Note that the vibration unit 451 may be attached to any position as long as vibration can be applied to the storage container 40. However, when provided on the outer surface of the wall part (in the example of FIG. 32, the bottom 41 and the side plate 43) of the storage container 40 in contact with the hair materials 100a, vibration can be applied to the hair materials 100a in the storage container 40 from the outside of the storage container 40 which does not interfere with the hair materials 100a, which is preferable. In particular, as exemplified in FIG. 32, when provided on the lower surface of the bottom 41, it is possible to effectively applies vibration to the hair materials 100a arranged in the storage container 40, so that it is possible to effectively improve the reduction in density of the hair materials 100a in the vicinity of the outlet 42 in the storage container 40.

### Deformation and the Like

The above-described embodiments are merely examples for carrying out the present invention. Therefore, the present invention is not limited to the above-described embodiments, and the above-described embodiments can be appropriately modified and implemented without departing from the gist thereof. In addition, the above-described embodiments can be appropriately combined as long as there is no contradiction.

For example, the first embodiment may be combined with the second embodiment and (or) the third embodiment. In this case, for example, when the protrusions 401 of the first embodiment are used, the first reciprocating mechanism 401a drives the protrusions 401 so that the shape of the protrusions 401 is matched with the shape of the bottom 41 in which the depressed portion 411 or 412 and any of the raised portions 421 to 423, 425 are formed and the tips of the protrusions 401 do not collide with the bottom 41.

Moreover, for example, the second embodiment and the third embodiment can be combined. Since the depressed portions 411 and 412 has a cross-sectional shape perpendicular to the longitudinal direction and the raised portions 421 to 423, and 425 have a cross-sectional shape parallel to the longitudinal direction, the bottom 41 has a complicated shape, but both can be combined without contradiction.

In addition, each of the fourth to sixth embodiments can be combined with other embodiments without any particular contradiction. Also, the vibration unit 451 of the sixth embodiment may apply vibration to a portion, which is in contact with the hair materials 100a, other than the storage container 40, such as the protrusions 401, the roller 402 and the restraining part 403 of the first embodiment, the pressing part 413 of the second embodiment, and the hair material driving part (roller) 432 of the fourth embodiment. Even in this case, it is possible to loosen the hair materials 100a to alleviate the bending and slackening of the hair materials 100a.

Further, the configuration of alleviating the bending and slackening of the hair materials 100a, which performs predetermined operations of the protrusions 401 and the roller 402 of the first embodiment, the storage container driving part 431 and the hair material driving part (roller) 432 of the fourth embodiment, the exhaust holes 442a of the fifth embodiment, and the vibration unit 451 of the sixth embodiment, etc., may be operated every time when the pulling hook needle 30 takes out the hair material 100a, or may be operated every time when the hair material 100a are taken out a plurality of times. Furthermore, in order to defend the hair material 100a from hindrance against being taken out by the pulling hook needle 30 or from being peeled, the operation of these members may be stopped while the pulling hook needle 30 is inserted from the outlet 42 into the storage container 40.

Moreover, in addition to the bottom 41 and the side plate 43 of the storage container 40, the protrusions 401, the roller 402 and the restraining part 403 of the first embodiment, the pressing part 413 of the second embodiment, the hair material driving part (roller) 432 of the fourth embodiment, and the like, which are in contact with the hair materials 100a, may be grounded and formed of a conductor. In this case, by removing static electricity of the hair materials 100a, when taking out one hair material 100a, the surrounding hair materials 100a can be prevented from moving together, so that it is possible to alleviate the bending and slackening of the hair materials 100a.

In addition, FIGS. 5 to 31 and FIGS. 23 to 32 showing the first embodiment and the third to sixth embodiments have illustrated the cases where the outlet 42 is formed from one side plate 43 to the other side plate 43 of the bottom 41, the outlet 42 may be formed only in a part in the horizontal direction perpendicular to the longitudinal direction (for example, similarly to FIGS. 15 to 21 shown for the second embodiment, the position where the pulling hook needle 30 is inserted and the periphery thereof).

The automatic hair transplant device described above can be described as follows.

The automatic hair transplant device includes a storage container that has an outlet at a bottom and has a hair material arranged on the bottom across the outlet, and an energizing part that applies a force including a component in a direction away from the outlet to the hair material arranged on the bottom of the storage container (first configuration). According to this configuration, by applying a force having a component in a direction away from the outlet to the hair material, the bending and slackening of the hair material are alleviated, and the reduction in density of the hair materials in the vicinity of the outlet in the storage container is improved. As a result, the pulling hook needle can stably catch the hair material at the outlet, so that the hair material can be stably transplanted.

In the first configuration, the energizing part may include protrusions that advance in a direction away from the outlet in a state where tips are inserted into a hair material bundle including a plurality of the hair materials arranged on the bottom of the storage container (second configuration). According to this configuration, since the tips of the protrusions advance along the hair materials, it is possible to apply a force in a direction away from the outlet to the hair materials (a force that alleviates bending and slackening of the hair materials).

In the second configuration, the protrusions may move in a direction approaching the bottom of the storage container so that tips of the protrusions are inserted into the hair material bundle, then advance in a direction away from the outlet, and then move in a direction away from the bottom of the storage container so that the tips are removed from the hair material bundle (third configuration). According to this configuration, it is possible to apply only a force that alleviates bending and slackening can be applied without applying a force that promotes the bending and slackening to the hair materials.

In the second or third configuration, when the protrusions advance in a direction away from the outlet in a state where the tips are inserted into the hair material bundle, the protrusions may be inclined so as to be away from the outlet as the protrusions are farther from the bottom (fourth configuration). According to this configuration, the protrusions can apply a force having not only a component in a direction away from the outlet but also a component in a downward direction to the hair materials. As a result, since the hair materials in the storage container are pressed toward the bottom side having the outlet, it is possible to effectively improve the reduction in density of the hair materials in the vicinity of the outlet in the storage container.

In the fourth configuration, the protrusions are in the inclined state from before the tips are inserted into the hair material bundle, and the tips are inserted into the hair material bundle by going straight along the inclined direction (fifth configuration). According to this configuration, since the inclination of the protrusions may not be changed, the mechanism for driving the protrusions can be simplified.

In any one of the second to fifth configurations, the energizing part may include a plurality of the protrusions whose moving direction is opposite in the state where the tips are inserted into the hair material bundle (sixth configuration). According to this configuration, it is possible to suppress the position of the hair material bundle from greatly varying due to the advancement of the protrusion.

In the sixth configuration, there may be a timing at which the plurality of protrusions whose moving direction is opposite in the state where the tips are inserted into the hair material bundle move simultaneously (seventh configuration). According to this configuration, by the advancement of the plurality of protrusions, the hair materials are pulled toward both end sides, so that the bending and slackening of the hair materials can be effectively alleviated.

In any one of the first to seventh configurations, the energizing part may include a roller whose conveying direction is a direction away from the outlet (eighth configuration). According to this configuration, it is possible to apply a force in a direction away from the outlet to the hair materials (a force that alleviates bending and slackening of the hair materials) by the rotation of the roller.

In the eighth configuration, the energizing part may include a plurality of the rollers whose conveying directions are opposite to each other (ninth configuration). According to this configuration, it is possible to suppress the position of the hair material bundle from varying by the rotation of the roller.

In the ninth configuration, there may be a timing at which the plurality of rollers whose conveying directions are opposite to each other rotate simultaneously (tenth configuration). According to this configuration, the hair materials are pulled toward both end sides by the rotation of the plurality of rollers, so that the bending and slackening of the hair materials can be effectively alleviated.

In any one of the first to tenth configurations, a restraining part that restricts the movement of the hair material bundle when the energizing part applies a force including a component in a direction away from the outlet to the hair material on a side opposite to the energizing part with the outlet interposed therebetween may be further included (eleventh configuration). According to this configuration, the bending and slackening of the hair materials can be effectively alleviated without moving the hair material bundle.

In any one of the first to eleventh configurations, the energizing part may vibrate in a state of being in contact with the hair material bundle (twelfth configuration). According to this configuration, since the hair materials can be loosened by the vibration of the energizing part, it is possible to alleviate the bending and slackening of the hair materials.

In any one of the first to twelfth configurations, the energizing part may be formed of a conductor in which a portion in contact with the hair materials is grounded (thirteenth configuration). According to this configuration, by removing static electricity of the hair materials, when taking out one hair material, the surrounding hair materials can be prevented from moving together, so that it is possible to alleviate the bending and slackening of the hair materials.

### REFERENCE CHARACTER LIST

120: Automatic hair transplant device
40: Storage container
41: Bottom
42: Outlet
43: Side plate
100a: Hair material
100b: Hair material bundle
401: Protrusion
402: Roller
403: Restraining part
411, 412: Depressed portion
413: Pressing part
421 to 423, 425: Raised portion
424: Inclined portion
431: Storage container driving part
432: Hair material driving part (roller)
441: Adhesive material
442a: Exhaust hole
442b: Exhaust part
451: Vibration unit

1. An automatic hair transplant device, comprising:
a storage container having an outlet at a bottom thereof, the storage container placing hair material across the outlet on the bottom; and
an energizing part configured to apply a force to the hair material placed across the outlet on the bottom of the storage container, the force having a component in a direction away from the outlet.

2. The automatic hair transplant device according to claim 1, wherein
the energizing part includes a protrusion that is configured to advance in the direction away from the outlet when a tip of the protrusion is inserted in a hair material bundle formed of the hair material placed on the bottom of the storage container.

3. The automatic hair transplant device according to claim 2, wherein the protrusion is configured to move in a direction approaching the bottom of the storage container to insert the tip into the hair material bundle, then advance in the direction away from the outlet, and then move in a direction away from the bottom of the storage container to remove the tip from the hair material bundle.

4. The automatic hair transplant device according to claim 2, wherein when the protrusion advances in a direction away from the outlet with the tip inserted in the hair material bundle, the protrusion is inclined such that the farther a position along the protrusion is from the bottom, the farther away the position is from the outlet.

5. The automatic hair transplant device according to claim 4, wherein the protrusion that is in an inclined state before the tip is inserted into the hair material bundle, is configured to travel straight along an inclined direction to be inserted into the hair material bundle.

6. The automatic hair transplant device according to claim 2, wherein the protrusion is one of a plurality of protrusions of the energizing part, each moving in an opposite direction when each tip of the plurality of the protrusions is inserted into the hair material bundle.

7. The automatic hair transplant device according to claim 6, wherein there is a timing at which the plurality of the protrusions move in the opposite direction simultaneously when the tips are inserted into the hair material bundle.

8. The automatic hair transplant device according to claim 1, wherein the energizing part includes a roller having a conveying direction away from the outlet.

9. The automatic hair transplant device according to claim 8, wherein the roller is one of a plurality of rollers of the energizing part, each having a conveying direction opposite to each other.

10. The automatic hair transplant device according to claim 9, wherein there is a timing at which the plurality of rollers rotate in the opposite direction to each other simultaneously.

11. The automatic hair transplant device according to claim 1, further comprising a restraining part that restricts movement of the hair material bundle when the energizing part applies the force to the hair material, the restraining part being on an opposite side to the energizing part with the outlet interposed in-between.

12. The automatic hair transplant device according to claim 1, wherein the energizing part is configured to vibrate while being in contact with the hair material bundle.

13. The automatic hair transplant device according to claim 1, wherein the energizing part is provided with a conductor having a grounded portion in contact with the hair material.
